(19) United States
(12) Patent Application Publication
(10) Pub. No.: US 2022/0051399 A1
(43) Pub. Date: Feb. 17, 2022

(54) METHOD AND SYSTEM FOR DETERMINING WELL-BEING INDICATORS

(71) Applicant: Health Partners PTE LTD, Singapore (SG)

(72) Inventor: Insu SONG, Singapore (SG)

(21) Appl. No.: 17/276,138

(22) PCT Filed: May 9, 2019
(86) PCT No.: PCT/SG2019/050446

§ 371 Date: Mar. 13, 2021

(51) Int. Cl. G06T 7/00; G06T 7/90; A61B 5/00; A61B 5/103; G16H 30/40; G16H 50/30

(52) U.S. Cl. CPC G06T 7/0012; G06T 7/90; A61B 5/0077; A61B 5/1032; A61B 5/441; A61B 5/7275; A61B 2576/02; G16H 30/40; G16H 50/30; G06T 2207/30088; G06T 2207/10024; A61B 5/7267; A61B 5/748

(57) ABSTRACT

The present invention relates to a method and system for determining well-being indicators. The well-being indicators comprise at least one of an antioxidant level, a stress level, a smoking level and a dietary level of fruits and vegetables. There is disclosed a method for determining well-being indicators from at least one skin image which comprises selecting at least one image element, wherein colour components disposed on the selected image element can be extracted, constructing colour histograms based on the extracted colour components, the colour histograms comprising prediction features which when extracted enable the well-being indicators to be determined from the skin images.

### FIELD OF THE INVENTION

The present invention relates to techniques for measuring shape and dispersion of colour histograms based on skin images and in particular but not exclusively, to a method and system for providing information on well-being by determining and analysing shapes and dispersions of colour histograms based on skin images.

### BACKGROUND TO THE INVENTION

The following discussion of the background to the invention is intended to facilitate an understanding of the present invention. However, it should be appreciated that the discussion is not an acknowledgement or admission that the material referred to was published, known or part of the common general knowledge in any jurisdiction as at the priority date of the application.

Functioning as the exterior interface of the human body with the environment, the skin is the most visible and the largest organ. The development of optical methods in the area of healthcare has stimulated investigation of optical properties of the human skin for various applications such as diagnosis of skin conditions and skin optical imaging, and the like. These applications in dermatology require the primary interactions of light with skin, hence requiring knowledge of optical properties of skin and subcutaneous tissues for interpretation and quantification of the diagnostic data.

While non-invasive methods exist for determining and monitoring skin health and skin colour, most also require measuring the difference between the reflected light signal and the reference light signal. A typical problem associated with these optical methods is that their effectiveness is often compromised by reflection of light by the skin and/or the reference light signal.

Conventional methods also require access to dermatological facilities where there are specialized equipment for various applications such as localization and pressurisation of the skin, light sources with specific wavelengths and spectrometers for measuring narrower band frequencies, and the like. As such, these conventional systems are often bulky and costly to manufacture.

Therefore, there is an urgent need for a cost-effective, efficient and effective method and/or system to address the aforementioned disadvantages. The present invention seeks to provide such a method and a system for determining antioxidant levels and/or other well-being indicators to overcome at least in part some of the aforementioned disadvantages.

### SUMMARY OF THE INVENTION

Throughout this document, unless otherwise indicated to the contrary, the terms “comprising”, “consisting of”, and the like, are to be construed as non-exhaustive, or in other words, as meaning “including, but not limited to”.

The present invention relates to a method and system for providing information on well-being based on skin images and in particular but not exclusively, to a method and system for providing information on a user's well-being by determining and analysing shapes and dispersions of colour histograms based on skin images of the user.

In accordance with a first aspect of the present invention, there is provided a method for determining well-being indicators from one or more skin images. The method comprises selecting one or more image elements based on selection parameters from the skin images, constructing colour histograms based on colour components values of the selected image elements, extracting prediction features from the colour histograms, the prediction features comprising central tendency, dispersion, shape and profile features of the colour histograms which can be used to provide information for various diagnostic tests.

Preferably, selecting the image elements comprises an iterative analysis of the image elements disposed on the skin image. The iterative analysis method comprises determining an average value for brightness and saturation, comparing the brightness and saturation values for each image element against the average value, wherein image elements within a predetermined range of values for brightness and saturation are selected.

Preferably, the constructed colour histograms comprise a distribution of number of pixels with brightness values within a predetermined range. The colour histograms can be constructed using colour component values of RGB, HSV, or HSL colour models or combinations of the component values.

Preferably, the prediction features comprise the values of mean, mode, standard deviation, skewness and kurtosis of the colour histograms, and combinations of the values.

Preferably, the well-being indicators can be at least one of antioxidant level, stress level, smoking level and dietary levels of fruits and vegetables.

In accordance with a second aspect of the present invention, there is provided a computer-readable medium comprising instructions which, when executed by a computer, causes the computer to carry out the steps for determining well-being indicators.

In accordance with a third aspect of the present invention, there is provided a device for determining well-being indicators from at least one skin image.

The present invention has at least the following advantages:

1. The present invention provides a new method for users to manage their physical health by conveniently taking images of their skin to provide information on their well-being.

2. The present invention advantageously provides higher accuracy and reliability in providing measurement readings by using clusters of points comprising similar properties of light intensity and colour component values, without the limitation of biased selection.

3. The present invention advantageously enables selecting multiple measurement points, hence providing convenience when used on a wide variety of skin surfaces, thereby not requiring focusing on specific points of the skin surfaces. Furthermore, measurements can be taken non-invasively and does not require pressurization, localisation or contact with skin surfaces.

4. The present invention advantageously compensates for differences in lighting conditions due to the surroundings; hence, it can be used with any camera, such as digital cameras or mobile phone cameras, and does not require specialized sources of light or impose restrictions on the light sources used.

5. The present invention advantageously can be integrated into a mobile diagnostic tool for providing onsite information on a user's lifestyle and diet, advantageously informing the user of their physical health within minutes.

Other aspects and advantages of the invention will become apparent to those skilled in the art from a review of the ensuing description, which proceeds with reference to the following illustrative drawings of various embodiments of the invention.

### BRIEF DESCRIPTION OF DRAWINGS

The present invention will now be described, by way of illustrative example only, with reference to the accompanying drawings, of which:

FIG. 1 is a schematic flow chart illustrating the method steps for determining well-being indicators in accordance with an embodiment of the present invention.

FIG. 2 is a schematic flow chart illustrating in more detail the method steps for determining well-being indicators in accordance with an embodiment of the present invention.

FIG. 3 is a schematic flow chart of the method steps of FIG. 2 illustrating the steps for pixel selection and analysis in accordance with an embodiment of the present invention.

FIG. 4 is a schematic diagram of the system for determining well-being indicators in accordance with an embodiment of the present invention.

FIG. 5 is a scatter plot illustrating the relationship between the actual antioxidant levels and the predicted antioxidant levels based on the prediction features of the colour histograms in accordance with an embodiment of the invention.

FIG. 6 is a schematic diagram illustrating the absorption curve of carotenoid antioxidants in the visible spectral region.

FIG. 7 is a diagram illustrating selection of pixels from an image of a palm in accordance with an embodiment of the present invention.

FIG. 8 and FIG. 9 are schematic diagrams illustrating respectively data histograms according to their measured components of red, blue and green for one participant with high antioxidant level and another participant with low antioxidant level.

### DETAILED DESCRIPTION

Particular embodiments of the present invention will now be described with reference to the accompanying drawings. The terminology used herein is for the purpose of describing particular embodiments only and is not intended to limit the scope of the present invention. Additionally, unless defined otherwise, all technical and scientific terms used herein have the same meanings as commonly understood by one of ordinary skill in the art to which this invention belongs.

The use of the singular forms “a”, “an”, and “the” includes both singular and plural referents unless the context clearly indicates otherwise.

The use of “or”, “/” means “and/or” unless stated otherwise. Furthermore, the use of terms “including” and “having” as well as other forms of those terms, such as “includes”, “included”, “has”, and “have”, are not limiting.

As used herein, the term “well-being” refers to a good or satisfactory condition of health, and the term “well-being indicator” is to be construed accordingly as a measure of a state of good or satisfactory condition of health, such as antioxidant levels, intake of vegetables, intake of fruits, stress levels, smoking levels, level of overall healthy diet and the like.

As used herein, the term “region of interest” refers to a cluster of image pixels or a plurality of such clusters that have been combined to form one region of image pixels that can be further processed to generate measurements with improved accuracy.

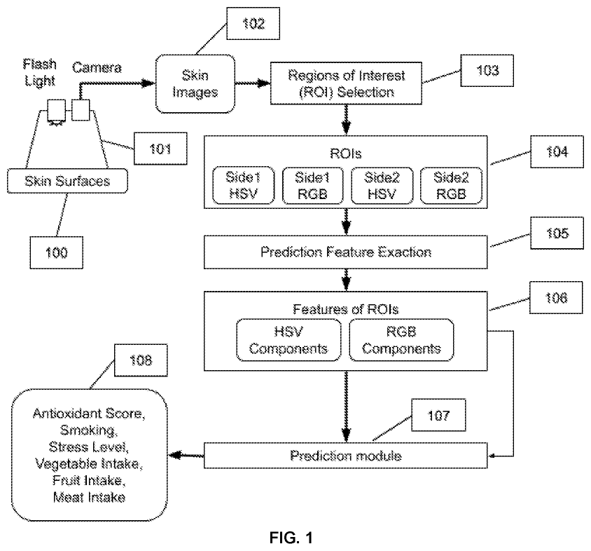

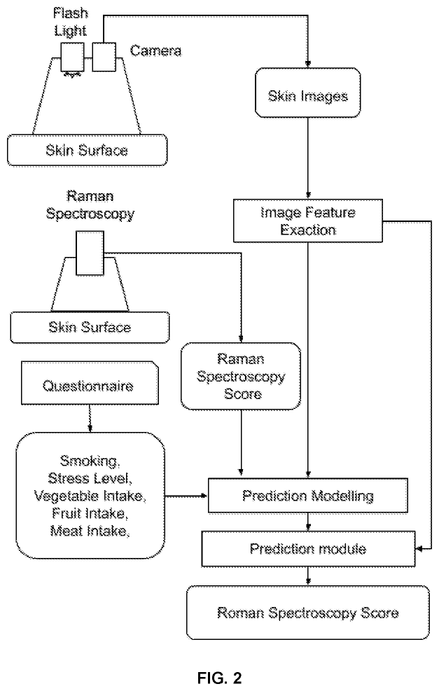

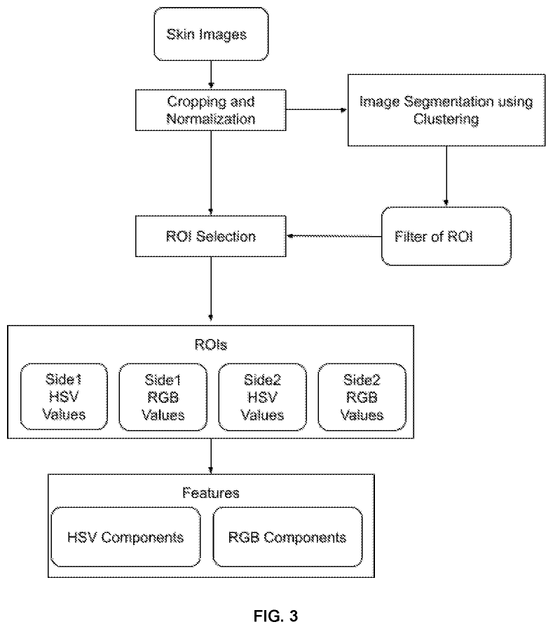

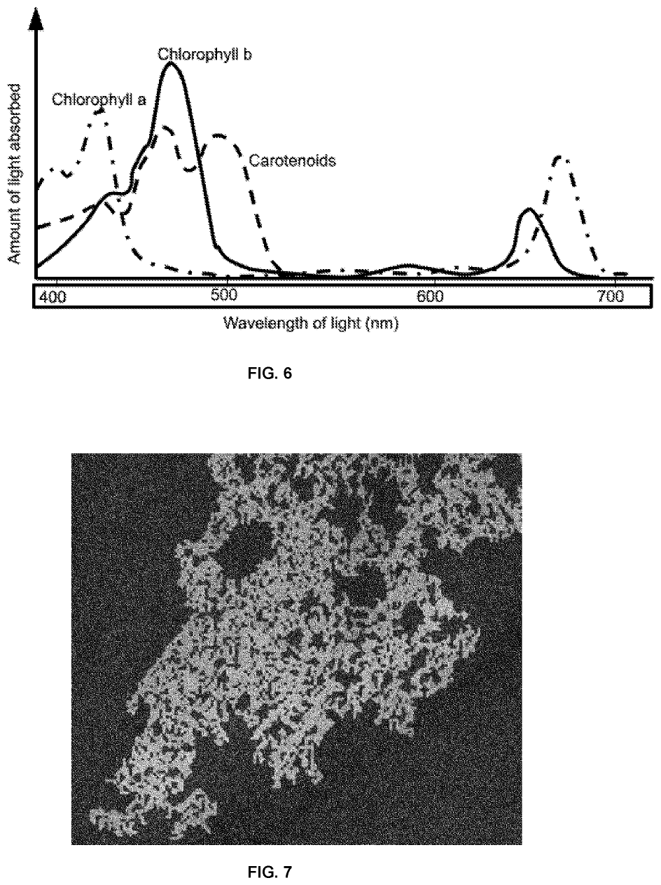

With reference to FIGS. 1 to 9, there is a method and system of determining well-being indicators in accordance with embodiments of the present invention. Firstly, a user takes one or more skin images from skin surfaces 100, 404 using a digital camera 101, 402. The skin images 102 are then transmitted to a processing unit 405 for processing. Thereafter, multiple points on the skin images are selected 103, 104 to obtain the measurements 105 using feature extractors 106 for determining the well-being indicators using a prediction model 107. In an embodiment of the invention, one or more combinations of the well-being indicators and/or the levels thereof are determined 108.

In an embodiment of the invention, there is a system and method for well-being determination and recommendation facility. This includes providing recommendations for improving well-being based on the well-being indicators and levels obtained.

Acquiring Images of Skin

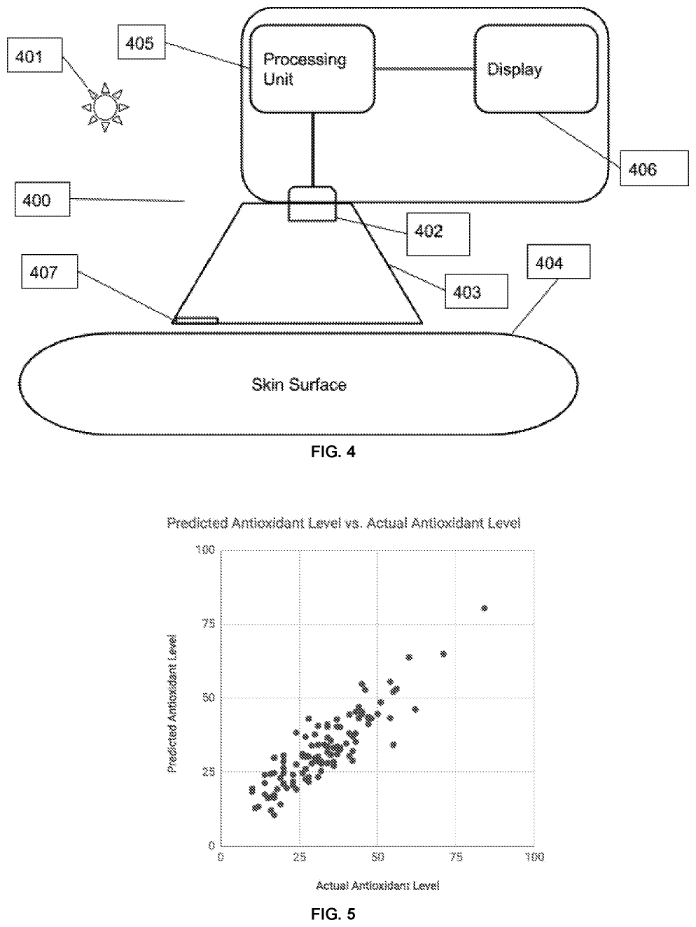

Referring to FIGS. 1 and 4, the user takes images of his skin surface 101, 407 using a camera 101, 402. The camera 101, 402 can be any digital camera or mobile camera. In one embodiment, the camera 101, 402 can be supported on the skin surface 404 by a guiding apparatus 403. This guiding apparatus 403 can allow better positioning of the camera 402 when taking images and focusing for enhanced quality skin images. One or more skin images 102 can be taken and stored on a memory storage device, before being transmitted for processing by a processor or processing unit 405.

Selection of Regions of Interest

According to some embodiments of the present invention, the regions of interest on the images are identified using histogram analysis and segmentation.

When taking images, the difference in lighting conditions as well as difference in skin colour can result in images reflecting different intensity of light. Therefore, according to some embodiments of the present invention, one or more regions of interest are selected on each image. This comprises identifying one or more regions on the image in which all image pixels in the region have at least one other neighbouring pixel and have similar lightness.

The images are analysed using colour histograms to detect an average brightness and saturation of each image. The average brightness and saturation values are used as a limit for selecting usable pixels, below which are pixels that are too dark, above which are pixels that are too bright. The pixels that are selected as usable are then processed via segmentation.

During segmentation, all the pixels in the image are scanned, preferably in one direction, either from left-to-right or right-to-left. A first pixel is selected and its brightness and lightness values measured. Thereafter, pixels adjacent to the first pixel are selected and their brightness and saturation values are also measured.

According to some embodiments of the present invention, while scanning, other similar pixels that are proximate and have similar brightness and saturation values are identified. Proximate pixels can include neighbouring pixels and those within a predetermined radius. These similar pixels are selected and merged to form a region of interest. The process continues iteratively by scanning all proximate pixels and merging all similar pixels until no further pixels merge to form the region of interest. It would be appreciated that the scanning process can be carried out iteratively for every n-th pixel in the image.

According to some embodiments of the present invention, a plurality of regions of interest (or clusters of image elements) are formed for the same image. Each region or cluster may have different values for average brightness and saturation. According to some embodiments of the present invention, the plurality of regions of interest are sorted based on the number of pixels in each region of interest, and the top N number of regions of interest can be selected for further processing.

Construction of Colour Histograms

According to some embodiments of the present invention, the selected regions of interest comprising image pixels (or image elements) are used to construct colour histograms by detecting and measuring the colour components.

According to some embodiments of the present invention, the colour components comprise the three primary components red, blue, and green of the RGB colour space. Each of the R, G, and B components are extracted for generating colour histograms.

According to some embodiments of the present invention, the primary colour components of the RGB colour space are converted into the Hue, Saturation, Value (HSV) colour space.

According to some embodiments of the present invention, HSV colour histograms are constructed from the RGB colour component values of the image pixels of the regions of interest.

According to some embodiments of the present invention, HSL colour histograms are constructed from the RGB colour component values of the image pixels of the regions of interest.

Extraction of Prediction Features from the Colour Histograms

According to some embodiments of the present invention, one or more prediction features are extracted from the colour histograms, wherein the prediction features comprise shape and profile features of the colour histograms which can be used to provide information for various diagnostic tests. It would be appreciated that spectroscopy based on colour histograms measures spectral distribution of the reflected light. Advantageously, unlike reflective spectroscopy, the present method of the invention does not require a reference light signal for comparison.

The prediction features are important datasets for generating prediction models, which , advantageously, provides improved accuracy in determining the general well-being acquired without requiring contact with skin surfaces. The prediction features can be generated by carefully selecting the pixels in the regions of interest to improve the reliability and accuracy of the prediction.

According to some embodiments of the present invention, the prediction features obtainable from the colour histograms are highly correlated with the skin's antioxidant levels. With reference to FIG. 5, a regression model constructed using the prediction features obtained has been found to be highly correlated with the measured antioxidant levels with R=0.882.

The generated colour histograms can be utilized to determine the well-being indicators. The prediction features of the image pixels extracted from carefully selected regions of interest provide valuable information for assessing physical well-being and dietary health.

To improve measurement accuracy and reliability, the carefully selected regions of interest can be filtered using the prediction features. This would be advantageous for images having oversaturated surfaces, areas that are not well-lit, images containing damaged skin surfaces or irregular textures, such as wrinkles and grooves which would affect the measurements of antioxidant levels on the skin. In one evaluation, the extracted image pixels from the carefully selected regions of interest were shown to improve the accuracy of the prediction model by over 15%.

According to some embodiments of the present invention, the prediction features from the colour histogram can comprise any one of the central tendencies (mean, mode, median), dispersion (standard deviation), and shapes (skewness and kurtosis). These features can be calculated using pixel intensity values from individual images, such as signal strength in a colour histogram.

According to some embodiments of the present invention, the prediction features from the colour histogram can comprise one or more numerical features derived from the signal intensities. The one or more numerical features can include generating variances between the discrete colour components, and further expressed as a function of any of the central tendencies, dispersion or shapes.

According to some embodiments of the present invention, the prediction features can be expressed as a function of the colour components. Alternatively, the prediction features can be expressed as a function of a difference between discrete colour components. The colour components can include red, green and blue (RGB) or Hue, Saturation and Value (HSV) or any suitable representations of the colour components.

According to some embodiments of the present invention, a plurality of prediction features can be generated as a numerical feature by taking differences between pixel intensity signals and/or other information derived from discrete colour components. It would be appreciated that variations in lighting conditions and skin colour are typically caused by melanin, which absorbs most of green light and blue light. It is known that melanin in the skin can interfere with measurements of carotenoids based on blue light.

The present invention advantageously eliminates the effects of environmental lighting conditions and differences in skin colours. Furthermore, reference lighting is not required for comparison. Following from a colour histogram, one of the colour components can be used as a reference. The colour components for any chosen pixel can be calculated as follows:

S_{RB}=S_{R}−S_{B}

S_{GB}=S_{G}−S_{B}

where S_{R} is the red component of the pixel, S_{G} is the green component of the pixel, S_{B} is the blue component of the pixel, S_{RB} is the difference between the red component and the blue component of the pixel, and S_{GB} is the difference between the green component and the blue component of the pixel.

Construction of Prediction Models

According to some embodiments of the present invention, one or more prediction model are built for estimating the levels of antioxidants and determining the user's well-being from the prediction features extracted from the colour histograms by the processing unit 405.

According to some embodiments of the present invention, the prediction models are constructed using a set of training data comprising a set of data having known characteristics, wherein the training data comprise diet and lifestyle data, measured antioxidant levels, and prediction features extracted from colour histograms of skin images of individuals.

According to some embodiments of the present invention, the training data comprise measurements of intake of food and drink over a period of two weeks and quantity of the same of an individual. Other lifestyle-related questions include well-being indicators, such as number of hours of exercise per week, the amount of alcohol consumption, frequency of smoking, and level of stress. For the same individual, measurements of the skin's antioxidant levels are taken using a Raman spectrometer and recorded. For the same individual, prediction features are also extracted from the colour histograms of the skin images. For example, the skin images can comprise one image of the palm and another from the back of the right hand.

According to some embodiments of the present invention, data transformation is performed on the training data by associating/correlating with known standard reference readings 407, such as a standard RGB colour chart. From the diet and lifestyle questionnaire, the total food consumption by each user over the last 2 weeks is determined based on each type of food. Antioxidant carotenoids are prevalent in most foods and can serve as an objective marker for fruit and vegetable intake, with reference to FIG. 7.

The training data are input into machine learning algorithms, such as support vector machine (SVM) for training purposes. The algorithm may be used in analysing data for classification and regression. To ascertain whether an optimal solution has been selected, a comparison of the prediction results of the algorithm with the predetermined values is carried out. The prediction results of the well-being indicators from the prediction features are compared with the lifestyle-related questions and the measurements of antioxidant levels.

The optimal solution yields desirable prediction models for determining antioxidant levels and other well-being indicators, such as intake of fruits, levels of exercise, levels of smoking, and levels of stress, based on the skin images. The models are trained using the training data set in order to identify the best matches between the skin images to the well-being indicators.

It would be appreciated that Naive Bayes Classifier (NB), Decision tree, Regression and other appropriate learning machine algorithms can be used for training purposes. According to some embodiments of the present invention, the prediction models can be built for a plurality of well-being indicators for providing information on physical health. These well-being indicators can include stress levels, smoking levels, intakes of fruits and vegetables, and the like.

For each well-being indicator, the respective models are trained to find the model coefficients using the test data comprising skin images, and the well-being indicator data, wherein the well-being indicator data include the numbers of hours of exercise per week, the amounts of alcohol consumption, the frequencies of smoking, the levels of stress, and antioxidant levels of the individuals. Using the test data, each of the models is trained to find the best matches between the skin images and the well-being indicators.

According to some embodiments of the present invention, the prediction models are used for analysing or identifying regions on a skin image to determine the well-being indicators.

Classification of Well-Being Levels

(i) Predictive measurement of the well-being indicator

According to some embodiments of the present invention, the method includes determining one or more numerical features as a predictive measurement for the well-being indicator for comparing with a reference measurement. The reference measurement can take the form of measurements taken using existing technology, such as Raman Spectroscopy.

The method includes determining one or more numerical features based on the colour components extracted from colour histograms and generating the well-being indicators as follows:

W=a₁×f₁+a₂×f₂+ . . . +a_{N}×f_{N}

wherein W is a well-being indicator, a are polynomial coefficients and fᵢ are the prediction features generated from the colour histograms. The method includes comparing the one or more numerical features with the reference measurements and classifying a plurality of pixels as belonging to at least one of multiple classes based on the predictive measurement.

In an embodiment of the invention, the default coefficients and exponents of the polynomials can be downloadable from a server for updates and localization. This allows users to upload and share the respective coefficients and exponents, thereby encouraging users with similar skin conditions and varying skin colours to benefit from these shared data points.

(ii) Quantitative measure of well-being based on numerical features

According to some embodiments of the present invention, the method includes determining one or more numerical features for comparing with a reference standard to derive a measure of well-being. This reference standard can be in the form of a scoring system to determine a user's well-being.

The method includes determining one or more numerical features based on the colour components extracted from colour histograms, comparing with a reference standard derived from diet and lifestyle data, deriving a score and/or measure of well-being based on the one or more numerical features, and providing quantitative measures of well-being indicators.

For example, the well-being indicators can be quantified using a scoring system along a scale of 0 to 4 or 5. Well-being indicators, such as stress levels, alcohol consumption and smoking levels, can be measured based on a score of 5 for most frequent (in terms of smoking) or highest (in terms of stress and alcohol consumption) and a score of 0 for no/least frequent (in terms of smoking) or no/lowest (in terms of stress and alcohol consumption).

(iii) Well-being levels based on binary classification

In another embodiment, the method can include determining one or more numerical features for evaluating well-being information. The method can include a binary classification for determining if a user is in good or poor health, or if the level of a well-being indicator is high or low.

The method can include determining one or more numerical features based on the colour components extracted from colour histograms, correlating with well-being indicators, and classifying the plurality of pixels as belonging to at least one of multiple classes based on the prediction values of the prediction models constructed using the training data and machine learning algorithms.

### EXAMPLES

### Example 1

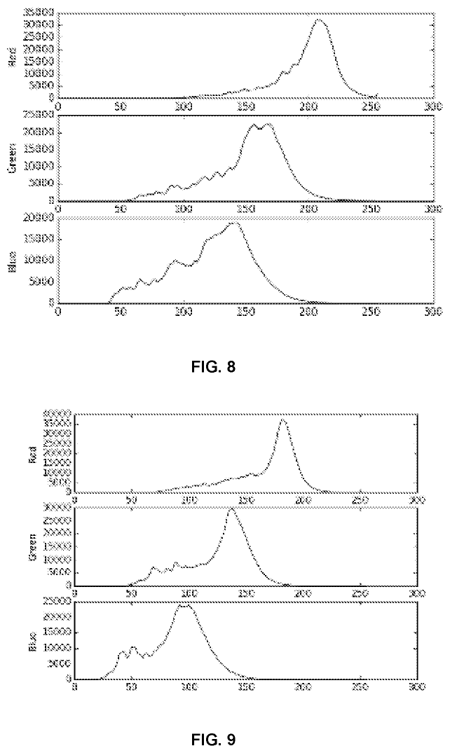

FIG. 8 illustrates the histograms of RGB components for one participant with high antioxidant levels, and FIG. 9 illustrates histograms of RGB components for one participant with low antioxidant levels. The differences in the values of the modes (i.e., the most frequent colour intensity component value for each colour histogram) derived from each of R, G and B component can be used to determine the levels of antioxidants in the human body. A comparison between the predictive measurement of well-being levels and the measured well-being levels is shown in Table I below.

TABLE I

| No. | S_{R}-S_{B} | Raman Spectroscopy |
|---|---|---|
| 1 | 100 | 37 |
| 2 | 60 | 10 |

### Example 2

In an evaluation, for an image size of 800×800, the light intensity was normalised and the image quality measured. An image region comprising 800×800 pixels of the skin image can be manually chosen.

To obtain quality regions of interest from the skin images for measuring well-being indicators, regions of interest comprising at least 400 pixels (20×20) were selected. Images containing at least one region of interest, each of which was large enough was selected for quality measurement purposes. Images without sufficient regions of interest were not used. In an embodiment of the invention, the pixels within the region of interest can be selected for further analysis. The regions of interest were sorted according to the number of pixels therein.

Thereafter, the top 5 largest regions of interest can be combined and used to plot the histograms of RGB and HSV components of the selected pixels.

### Example 3

To evaluate precision in using prediction features of the colour histograms, the lifestyle and eating habits data were used to build prediction models of the antioxidant levels. Accordingly, the total consumption for each item of food was summed for the prediction of the antioxidant levels.

For this evaluation, classification (prediction) models were constructed using SVM (Support Vector Machine) and NB (Naive Bayes Classifier). The classification models was evaluated with 10-fold cross-validation. For the classification task, instances were divided into two classes, including high and low antioxidant levels, based on the value of the Raman Spectroscopy score.

The lifestyle and eating habit data and the prediction features of the colour components were used to classify the antioxidant levels in the body into high or low levels. A comparison of the classification results is displayed in Table II below.

TABLE II

| | | | Prediction | Prediction |
|---|---|---|---|---|
| | Prediction | Prediction | using Features | using Features |
| | using | using | of Colour | of Colour |
| | Lifestyle Data | Dietary Data | Histograms | Histograms |
| | (using NB) | (using NB) | (using NB) | (using SVM) |
| Sensitivity | 0.61 | 0.69 | 0.70 | 0.69 |
| Specificity | 0.61 | 0.69 | 0.70 | 0.69 |
| Precision | 0.61 | 0.71 | 0.71 | 0.70 |
| F-Measure | 0.609 | 0.69 | 0.70 | 0.69 |
| ROC Area | 0.619 | 0.61 | 0.70 | 0.69 |
| PRC Area | 0.612 | 0.67 | 0.69 | 0.63 |

Table II shows that each of the prediction features—lifestyle data, dietary data and colour histograms—were informative in estimating antioxidant levels: the rates were significantly above 0.5 (50%).

### Example 4

For regression, SPSS can be used for building and analysing the model with a confidence interval of 0.05%. A regression model was fit to predict antioxidant levels using the extracted prediction features of colour histograms. The predicted values were then compared to the actual values (obtained from the questionnaire containing the lifestyle and diet data) by calculating correlations. The average, mode, median, standard deviation, skewness and kurtosis values of the colour histograms, and the differences between them were used to construct the linear regression models.

FIG. 5 illustrates a scatter plot for analysing the relationship between the actual antioxidant levels and the predicted antioxidant levels based on the prediction features of the colour histograms. The predicted antioxidant levels are highly correlated with the actual antioxidant levels with R=0.882 and R²=0.778. Thus, the regression model of the present invention has significant potential for predicting antioxidant levels.

Table III shows some of the prediction features and their respective correlations to actual antioxidant levels. Linear regression analysis was conducted for significance p <0.05. The parameters of the regression include the mean, median, mode, skewness and kurtosis. GB and RB are representations of prediction features of colour histograms. GB is derived based on the difference operator of a green colour histogram and a blue colour histogram; RB is derived based on the difference operator of a red colour histogram and a blue colour histogram.

TABLE III

| No. | Prediction Features | R Value (Correlation) |
|---|---|---|
| 1 | Mean GB | 0.46 |
| | Mean RB | 0.45 |
| 2. | Median RB | 0.44 |
| | Median GB | 0.43 |
| 3. | Mode RB | 0.41 |
| 4. | Skewness B | 0.30 |
| | Skewness RB | 0.161 |
| 5. | Kurtosis B | 0.133 |

The method according to some embodiments of the present invention is implemented in program instruction form that can be executed by various computer means to be recorded in computer-readable media. The media may also include, alone or in combination with the program instructions, data files, data structures and the like. The media and program instructions may be those specially designed and configured.

Examples of computer readable recording media include optical recording media, floppy disks and hardware devices that are specially configured to store the program instructions.

The present invention can be integrated into a mobile diagnostic tool for providing onsite information on a user's lifestyle and diet, advantageously informing the user of their physical health within minutes. FIG. 4 describes a mobile diagnostic tool and method for a user to be informed of their physical health onsite in accordance with an embodiment of the present invention. The mobile diagnostic tool 400 comprises a communication device. A communication device includes a computing device and a mobile computing device, such as a mobile phone, tablet, laptop or personal digital assistant. In this embodiment, the user unit is in the form of a mobile device.

The mobile device 400 can be positioned on a guiding apparatus 403 atop a user's skin surface. The guiding apparatus 403 allows accurate positioning of the mobile device towards a target area on the skin. The guiding apparatus allows illumination of light on the skin surface from a light source 401, which can be placed inside the guiding apparatus 403 or outside of the apparatus 403. The light source 401 can be any broad spectrum lighting, such as sunlight, LED flashlights and the like. The mobile device comprises at least a common RGB digital image sensor 402 that can be found on many mobile devices. In another embodiment of the present invention, the images can be taken by hand and without the use of the guiding apparatus.

Thereafter, one or more images of the skin surfaces can be taken and sent to the processing unit of the mobile device for processing. In a first step, one or more image pixels disposed on the skin images are selected and extracted, with the extracted image pixels comprising colour components. Alternatively, clusters of image pixels disposed on the skin images can be selected and extracted. In both instances, the processing unit constructs colour histograms based on the extracted colour components, the colour histograms comprising prediction features which when extracted enable the well-being indicators to be determined from the skin images.

It is to be understood that the above embodiments have been provided only by way of exemplification of this invention, and that further modifications and improvements thereto, as would be apparent to persons skilled in the relevant art, are deemed to fall within the broad scope and ambit of the present invention described herein. It is to be understood that features from one or more of the described embodiments may be combined to form further embodiments.

1. A method for determining one or more well-being indicators from one or more skin images, comprising:
selecting one or more regions of interest (Rats) from the images, based on one or more selection parameters, wherein each ROI comprises a plurality of image elements, and the selection parameters comprise ranges of acceptable lightness, colour saturation, and colour component values;
constructing one or more colour histograms from at least one of the regions of interest, wherein the colour histograms comprise RGB colour component histograms, HSV colour component histograms and HSL colour component histograms;
extracting one or more prediction features from at least one of the colour histograms, wherein the prediction features comprise mean; mode, median, standard deviation, kurtosis and skewness values of the colour histograms; determining one or more well-being indicators from the prediction features.

2. The method according to claim 1, wherein the prediction features further comprise at least one of the differences between mean, mode, standard deviation, kurtosis and skewness values of the colour histograms. (currently amended) The method according to claim 1, wherein the step of determining well-being indicators further comprises processing the prediction features based on the following formula:
W=a₁×f₁+a₂×f₂+ . . . +a_{N}×f_{N}
wherein W is a well-being indicator, a; are polynomial coefficients and f: are the prediction features generated from at least one of the colour histograms.

4. The method according to claim 1, wherein the step of selecting the regions of interest further comprises selecting similar image elements that are adjacent to one another and are similar based on at least one of colour component values of the image elements.

5. The method according to claim 1, further comprising an iterative analysis of adjacent image elements and lightness measurements for selecting similar image elements.

6. The method according to claim 1, wherein the analysis of the adjacent image elements further comprises the steps of: determining average values for brightness and saturation for each of adjacent image elements by generating colour histograms; determining the brightness and saturation values for each of the adjacent image elements;
comparing the brightness and saturation values for each of the adjacent image elements; and
extracting each of the adjacent image elements which are within relevant tolerance values for brightness and saturation based on the selection parameters.

7. The method according to claim 1, further comprising selecting a plurality of regions of interest comprising similar pixels having similar lightness for eliminating different lighting intensities of the one or more skin images.

8. The method according to claim 1, further comprising filtering the one or more regions of interest using any one of the prediction features, wherein regions comprising colour inconsistencies and texture unevenness are filtered out.

9. The method according to claim 1, further comprising acquiring the one or more skin images from at least of the following:
images taken by mobile phone cameras, digital cameras and the like, images transmitted to mobile devices, communication devices and the like.

10. The method according to claim 1, wherein the well-being
indicators comprise at least one of an antioxidant level, a stress level, a smoking level and a dietary level of fruits and vegetables.

11. The method according claim 1, further comprising the measurement of colour components of each similar image element and comparing intensities for each of the colour components, wherein one or more significant differences between the colour component intensities determines the well-being indicators, using a regression function taking the significant differences as independent variables.

12. The method according to claim 11, wherein the colour components comprise at least one of the following: Red, Green and Blue of the RGB colour model, Hue, Saturation and Value of the HSV colour model, and Hue, Saturation and Lightness of the HSL colour model.

13. The method according to claim 3, wherein the prediction features further comprise at least one of the differences between the prediction features and previous prediction features.

14. A computer-readable storage medium comprising instructions which, when executed by a computer, cause the computer to carry out the steps for determining one or more well-being indicators, comprising:
selecting a plurality of measurement points from which the well-being indicator can be determined;
extracting similar pixels disposed on the plurality of measurement points to obtain one or more regions of interest;
constructing one or more colour histograms from at least one of the regions of interest, wherein the colour histograms comprise RGB colour component histograms, HSV colour component histograms and HSL colour component histograms;
extracting one or more prediction features from at least one of the colour histograms, wherein the prediction features comprise mean, mode, median, standard deviation, kurtosis and skewness values of the colour histograms;
determining one or more well-being indicators from the prediction features from at least one of the skin images.

15. The computer-readable storage medium according to claim 14, wherein the
prediction features further comprise at least one of the following: differences between each of mean, mode, median and kurtosis and skewness and HSV components.

16. The computer-readable storage medium according to claim 14, wherein the predictive measurement of well-being further comprises processing the prediction features based on the following formula:
W=a₁×f₁+a₂×f₂+ . . . +a_{N}×f_{N}
where a; are polynomial coefficients and f, are the prediction features generated from the
one or more regions of interest.

17. The computer-readable storage medium according to claim 14, wherein the similar pixels comprise one or more adjacent pixels having similar lightness.

18. The computer-readable storage medium according to claim 14, further comprising:
determining average values for brightness and saturation for each of the adjacent pixels by generating colour histograms; determining the brightness and saturation values for
each of the adjacent pixels; comparing the brightness and saturation values for
each of the adjacent pixels; and
extracting each of the adjacent pixels which are within the relevant tolerance values for brightness and saturation.

19. The computer-readable storage medium according to claim 14, further comprising selecting a plurality of regions of interest comprising similar pixels having similar lightness for eliminating different lighting intensities of the one or more skin images.

20. The computer-readable storage medium according to any one of claim 19, further comprising acquiring the one or more skin images by use of at least of the following: images taken by mobile phone cameras, digital cameras, images transmitted to mobile devices, communication devices and the like.

21. (canceled)

22. (canceled)

23. The computer-readable storage medium according to claim 14, wherein the prediction features further comprise at least one of the differences between the prediction features and previous prediction features.
